(19) United States
(12) Patent Application Publication
(10) Pub. No.: US 2010/0178658 A1
(43) Pub. Date: Jul. 15, 2010

(54) SCREENING ASSAYS FOR INHIBITORS OF BETA AMYLOID PEPTIDE ION CHANNEL FORMATION

(75) Inventors: Michael Mayer, Ann Arbor, MI (US); Jerry Yang, La Jolla, CA (US)

Correspondence Address: HARNESS, DICKEY & PIERCE, P.L.C., P.O. BOX 828, BLOOMFIELD HILLS, MI, 48303, US

(73) Assignee: The Regents of the University of Michigan, Ann Arbor, MI (US)

(21) Appl. No.: 12/602,373

(22) PCT Filed: May 30, 2008
(86) PCT No.: PCT/US08/06885

§ 371 Date: Nov. 30, 2009

Related U.S. Application Data
(60) Provisional application No. 60/932,293, filed on May 30, 2007.

(51) Int. Cl. G01N 33/567

(52) U.S. Cl. 435/7.21

(57) ABSTRACT

Screening assays and methods of employing the screening assays designed to identify potential inhibitors of amyloidal neurodegenerative disease. The screening assays comprises providing a membrane construct disposed on a substrate and contacting the membrane construct with Aβ peptide capable of forming an Aβ peptide ion channel in the construct. The membrane construct is then contacted with a test compound. Aβ peptide ion channel activity is determined after the construct has incubated with the Aβ peptide in the presence and in the absence of the test compound. A reduction in the Aβ peptide ion channel activity of the membrane construct contacted with the test compound in comparison to a different membrane construct contacted with the same Aβ peptide in the absence of said test compound indicates that the test compound is an inhibitor of amyloidal neurodegenerative disease.

### FIELD

The present disclosure relates to functional assays to identify test compounds which can inhibit the neurotoxic ion channel activity of Beta-Amyloid peptides (Aβ peptides).

### BACKGROUND AND SUMMARY

The statements in this section merely provide background information related to the present disclosure and may not constitute prior art.

Amyloid protein misfolding has been shown to be the direct cause of a number of highly devastating neurodegenerative diseases such as Alzheimer's disease, Parkinson's disease, and Creutzfeldt-Jacob syndrome.

Alzheimer's disease (AD) is the most common neurodegenerative disorder in the elderly and affects 4.5 million people in the US and 25 million people worldwide. AD is the fourth leading cause of death in industrialized societies (exceeded only by heart disease, cancer, and stroke) and is the third most expensive disease in the US (with a cost of over $100 billion annually). The number of Americans with AD is expected to rise to as many as 13 million by the year 2050, underscoring the need for developing new methods for treatment of this debilitating disease. Current treatments for AD provide only modest, temporary, and palliative benefits—AD invariably leads to progressive dementia, disability, and death.

One of the hallmark characteristics of AD is the accumulation of amyloid plaques in the brain. The major component of the amyloid plaques is a peptide (molecular weight ˜4 kD) made up of 40 or 42 amino acids—referred to as β-amyloid (or Aβ) peptide—that aggregates into insoluble fibrillar structures. Aβ peptide is a fragment of a transmembrane protein called amyloid precursor protein (APP), and the extracellular release of this peptide requires the cleavage of APP by β- and γ-secretases in the membrane. Although the formation and removal of Aβ peptides are normal neurophysiological processes, accumulation of Aβ peptides in the brain leads to deposition of β-amyloid plaques.

The most probable cause for the development of AD is the interaction of Aβ peptides, oligomers, and/or fibrils with cellular components in the brain. This so-called “amyloid hypothesis” is supported, in part, by the finding that aggregated Aβ is toxic to cultured neurons, although there is still much debate as to which form of aggregated Aβ (e.g. oligomers or fibrils) is most toxic. Oligomers of Aβ peptides have been found to generate pore-like structures in cellular membranes that lead to acute electrophysiological changes and neuronal dysfunction in Alzheimer's disease. Solutions of Aβ fibrils have also been found to induce ion channel activity. Aggregated Aβ can interact directly with cellular proteins and attenuate their activity. In addition, increasing evidence shows that Aβ oligomers and Aβ fibrils destabilize membrane potentials and disrupt calcium homeostasis in neurons through the formation of ion channels.

Ion channel disruption has been studied in a different context with the use of anti-microbial peptides, for example, alamethicin and provides that the mechanism of action includes disruption of ion channel activity through pore disruption. Yu, L. et al. Biochem & Biophys. Acta (2005). 1716: 26-39 and Li, P. et al. J. Biol. Chem. (2004), 48(11): 50150-50156.

One hypothesis for the deleterious effects of oligomers of Aβ peptides is based on the generation of pore-like structures in cellular membranes that lead to acute electrophysiological changes and neuronal dysfunction in AD (solutions of Aβ fibrils have also been found to induce ion channel activity) (Hartley, D. M. et al. Journal of Neuroscience 19, 8876-8884 (1999); Lin, H., Bhatia, R. & Lal, R. Faseb J 15, 2433-44 (2001); Novarino, G. et al. J Neurosci 24, 5322-30 (2004)).

Hence there is a need for rapid, simple and reproducible screening assays to identify lead compounds having inhibitory activity against pathogenic Aβ oligomers and Aβ fibrils, which can be performed in high-throughput fashion.

Further areas of applicability will become apparent from the description provided herein. It should be understood that the description and specific examples are intended for purposes of illustration only and are not intended to limit the scope of the present disclosure.

### DRAWINGS

The drawings described herein are for illustration purposes only and are not intended to limit the scope of the present disclosure in any way.

FIG. 1. Illustration of the Fluorescence Correlation Spectroscopy (FCS) assay to measure ion channel activity in membrane constructs having Aβ peptide ion channels. Fluorophores escape from preloaded liposomes can be detected from free flurophore based on the differences in diffusion coefficients between the entrapped fluorophore in liposomes versus the free fluorophore in the solution.

FIG. 2. Inhibition of Aβ peptide ion channel activity using the Fluorescence Correlation Spectroscopy (FCS) assay. (A) At time t0, rhodamine 6G in liposomes diffuses as a large fluorescent particle with diffusion time τ1 ˜6 ms. (B) At time t1, after pore-forming peptide is added, partial release of rhodamine 6G into solution occurs from the vesicles resulting in a diffusion time of free rhodamine of τ˜47 μs. (C) At time t2, when most of the rhodamine fluorophore will have leaked out of vesicles through Aβ pores. FCS will detect diffusion times of 55 μs predominantly from released rhodamine. (D) The same fluorophore release mechanism can be used for a functional screening of small molecules inhibiting the pore forming activity of Aβ oligomers. (E) Small potential inhibitor molecules will be added shortly before (or simultaneously) to the addition of Aβ oligomers. (F) An effective inhibiting molecule will prevent pore formation. In this case, FCS will only detect the diffusion time of the rhodamine entrapped in slowly moving liposomes instead of fast moving free rhodamine. Note the expected significant different scale in the y-axis of the correlation function G(τ) between (C) and (F).

FIG. 3. Inhibition of Aβ peptide ion channel formation after addition of nicotine in a molar ratio of 1:1. Panel A: Addition of 37 μM Aβ peptide concentration in the absence of nicotine resulted in pore forming activity and the presence of pores. Panel B: Addition of the same Aβ peptide concentration in addition to 37 μM of nicotine eliminated ion channel pore forming activity during the test period.

FIG. 4. Panel A: Disruption of Aβ ion channels formed in the presence of Aβ peptide (1-42) and no test compound. Panel B: Addition of nicotine at 2-fold molar excess with respect to Aβ peptide (1-42) disrupted preformed ion channels Panel C: Addition of nicotine at 4-fold molar excess with respect to Aβ peptide (1-42) disrupted preformed ion channels almost completely within 10 min.

FIG. 5. Inhibition of Aβ ion channel formation by Tannic acid in a molar ratio to Aβ peptide of 1:1. A) 30 minutes after the addition of 37 μM Aβ peptide concentration in the absence of Tannic acid resulted in pore forming activity and the presence of pores. B) 90 minutes after the addition of 37 μM Aβ peptide concentration in the presence of Tannic acid did not result in Aβ peptide during the entire test period of 90 minutes.

### DETAILED DESCRIPTION

The following description is merely exemplary in nature and is not intended to limit the present disclosure, application, or uses.

The present teachings provide for novel functional screening assays for inhibitors of beta-Amyloid (Aβ) peptide ion channel activity. In the present teachings, artificial and synthetic membrane structures, for example liposomes, unilamellar vesicles, micelles, and lipid bilayers and biological membranes, for example, whole cells, cell membranes and fragments thereof, can be utilized to functionally incorporate neurotoxic Aβ peptide containing ion channels. Assays of the present teachings incorporate the natural and/or synthetic membrane structures having Aβ peptide ion channels as a model for amyloidal neurodegenerative disease processes, including Alzheimer's disease, Parkinson's disease, Huntington's disease, Down's Syndrome, spongiform encephalopathies, including, Creutzfeldt-Jacob Syndrome, Bovine Spongiform Encephalopathy, and Kuru. Therapeutic lead compounds for the treatment of amyloidal neurodegenerative diseases can be identified by screening test compounds that can selectively decrease the formation and activity of Aβ peptide ion channels in membrane constructs.

The functional assays of the present teachings can be used to determine whether selected small molecules or test compounds can inhibit the neurotoxic Aβ ion channel activity in liposomes, unilamellar vesicles, micelles, lipid bilayers, and living cells, for example, neuronal cells derived from a cell culture line or isolated from brain tissue in an animal, including mice and human. In some embodiments, methods for identifying inhibitors of amyloidal neurodegenerative disease can include: providing a membrane construct disposed on a substrate; contacting the membrane construct with Aβ peptide capable of forming an Aβ peptide ion channel in the construct; contacting the membrane construct with a test compound; determining an Aβ peptide ion channel activity after the construct has incubated with the Aβ peptide in the presence of said test compound; wherein a reduction in the Aβ peptide ion channel activity of the membrane construct contacted with the test compound in comparison to a different membrane construct contacted with the Aβ peptide in the absence of the test compound indicates that the test compound is an inhibitor of amyloidal neurodegenerative disease.

U.S. Provisional Patent Application Ser. No. 60/940,869 filed May 30, 2007 by University California San Diego, entitled “Compounds and Methods for the Diagnosis and Treatment of Amyloid Associated Diseases” is referred to and incorporated by reference in its entirety.

### Membrane Constructs

In various screening assays in accordance with the present teachings, membrane constructs can be used to provide the physiological structure for the formation and inclusion of Aβ peptide ion channels. The functionality of Aβ peptide ion channels involved in the transport and/or energy transduction requires a membrane. The present teaching provides for assays employing several different classes of membrane constructs that utilize Aβ peptide ion channels to simulate neurotoxic Aβ peptide ion channels in living neuronal cells. Membrane constructs having functional Aβ peptide ion channels embedded in the membrane are then used to screen for molecules that can decrease or inhibit the formation, function or activity of these Aβ peptide ion channels.

As used herein, membrane constructs can include any form of model membrane systems capable of incorporating an Aβ peptide ion channel, for example, planar lipid bilayers, multilamellar liposomes, unilamellar liposomes, unilamellar vesicles, proteoliposomes, micelles, mixed detergent-lipid-micelles, isolated primary neurons and cell cultured brain cell lines, whole cell membranes (as found in viable eukaryotic and prokaryotic cells, for example any cell derived from brain tissue from any animal, including human and laboratory animals, such as neuronal cells, (neurons), astrocytes, oligodendrocytes, glial cells and the like), cell membrane fragments and mixtures thereof. As used herein, neuronal cells can include any neuron or neuron like cell found in any part of the brain from any animal. A cell cultured brain cell is a tissue cultured cell whose origin is from brain tissue, for example, SH-SY5Y neuronal cells (human neuroblastoma cells).

In some embodiments, membrane constructs for use in the screening assays of the present teachings can include planar lipid bilayers, i.e. artificial lipid bilayer membranes having Aβ peptide ion channels embedded in the bilayer. Methods for making lipid bilayer membranes are well known in the field of membrane electrophysiology. A bilayer lipid membrane can be formed from two layers of amphiphilic lipid molecules for example, phospholipids, sterols, and glycolipids in which one part of the molecule is hydrophilic and the other part is lipophilic. The polar groups are in contact with water and the hydrocarbon chains of the lipids are oriented away from the water. Hydrophobic parts of the lipid molecules aggregate thus forming the self-assembled lipid bilayers in aqueous solutions. The construction and use of tethered planar lipid bilayered membranes have been described in, for example, Mayer et al., (2006); Trojanowicz, M. “Miniaturized biochemical sensing devices based on planar bilayer lipid membranes”, (2001) Fresenius J. Anal. Chem. 371:246-260 and Darszon, A. “Strategies in the reassembly of membrane proteins into lipid bilayer systems and their functional assay”, (1983) J. Bioenergetics and Biomembranes, 15(6):321-334.

Membrane constructs using planar lipid bilayers are well suited for electrophysiological measurement of ion channel activity, in particular, for use with self-assembled Aβ peptide ion channels.

In some embodiments, the lipid bilayered membrane can be made of 50% palmitoyloleoyl-phosphatidylethanolamine (POPE) and 50% palmitoyloleoyl-phosphatidylglycerol (POPG) in electrolyte containing 100 mM K₂HPO₄ at pH 6.5.

In still further embodiments, planar lipid bilayers can be made using the “folding technique.” A Teflon film (Eastern Scientific Inc., Osterville, Mass., USA) with a pore diameter 0.002-0.25 mm can be pretreated on each side with 2.5 μL of 5% hexadecane in pentane and air dried. This film can be mounted using vacuum grease for example, high vacuum grease (Dow Corning, Midland, Mich., USA) to a custom made Teflon chamber separating two buffer compartments each with a volume capacity of about 0.005 mL to about 5 mL. After adding about 1 mL of electrolyte (0.01 M to 1.00 M KCl buffered with HEPES pH 7.4) to each compartment, lipids can be spread from a solution in pentane onto the surface of the electrolyte solutions (specifically, 4-6 μL from 25 mg mL⁻¹ solution DiPhyPC or from 6.25 mg mL⁻¹ each of 50% DOPS and 50% POPE or from 25 mg mL-1 DODAP mixed with 25 mg mL⁻¹ DiPhyPC in a 1:9 ratio. 3 additional mL of electrolyte solution can be added to each side of the chamber to raise the liquid levels above the aperture.

In some embodiments, planar lipid bilayers can be formed from apposition of two monolayers of lipids using the method described by Montal et al. (1972), Proc. Natl. Acad. Sci. USA. 69:3561-3566. In some embodiments, the planar lipid bilayers can be obtained by consecutively raising the liquid level in each compartment until the pore was completely covered by electrolyte. If at this point the pore is not closed by a lipid bilayer, then the liquid level in one or both compartments is lowered below the pore level by aspirating electrolyte into a syringe, followed by raising the electrolyte solution again. This cycle can be repeated until a bilayer is obtained that has a minimum capacitance of 70 pF, and until the resulting membrane is stable (i.e., no significant current fluctuations above the baseline noise level) in the range of ±200 mV applied potential for several minutes.

In some embodiments, planar lipid bilayers can also be made using the “painting technique”.(Mueller et al., (1962), Circulation. 26:1167-1171). Each side of a pore in a bilayer cup, for example, a Delrin perfusion cup, volume 1 mL, and pore diameter 250 μM (Warner Instruments LLC, Hamden, Conn., USA) can be pretreated with about 2 μL of a 25 mg mL⁻¹ solution of DiPhyPC in hexane. After adding recording buffer (0.01 M KCl to 1.00 M KCl buffered with HEPES, pH=7.4) to both compartments of the bilayer setup, a solution of 20 mg mL⁻¹ DiPhyPC in n-decane is “painted” over the pore by using a paint brush with a fine tip. The thinning of the decane droplet can be followed to form a planar lipid bilayer by monitoring the capacitance of the bilayer. In case the decane droplet does not thin out spontaneously, bubbled air can be introduced in the chamber underneath the pore. The rise of these air bubbles in the vicinity of the pore usually helps to thin out the decane/lipid droplet. After verifying that bilayers are stable for several minutes after an applied voltage across the bilayer (in the range of ±200 mV applied voltage) and that the capacitances are above 80-90 pF, by addition of (4-8 μL from 1 ng mL-1 in ethanol, or other concentrations in another appropriate solvent), (−) nicotine, dopamine, tannic acid, curcumin, salicylic acid, L-(−)-norepinephrine (+)-bitartrate salt monohydrate, L-DOPA, N-methyl dopamine hydrochloride, BTA-EG4, BTA-EG6 can be added directly to the bilayer chambers.

In some embodiments, methods for making and using membrane constructs, including lipid bilayer membranes, vesicles, and liposomes can be found in Darszon, A., “Strategies in the reassembly of membrane proteins into lipid bilayer systems and their functional assay” (1983). J. Bioenergetics and Biomembranes, 15(6):321-334; and Mayer, M., et al., in Biosensors: A practical approach (eds. Cooper, J. M. & Cass, A. E. G.) 153-184 (Oxford University Press, Oxford, 2003), both of these references are incorporated herein in their entireties.

### Amyloid-Beta (Aβ) Peptides

The hallmark characteristic of an amyloidal neurodegenerative disease, for example, Alzheimer's disease is the accumulation of β-amyloid plaques in the brain. The major component of the amyloid plaque is a peptide made up of 40 or 42 amino acids, referred to as Aβ(1-40) or Aβ(1-42) peptides. These Aβ peptides aggregate into insoluble fibrillar structures and into soluble oligomers some of which form pores in lipid bilayers and in cell membranes. Although the formation and removal of Aβ peptides are normal neurophysiological processes, accumulation of Aβ peptides in the brain leads to deposition of β-amyloid plaques. An increasing body of literature shows that aggregated Aβ is toxic to cultured neurons, and that Aβ oligomers destabilize membrane potentials and disrupt calcium homeostasis in neurons.

Electrophysiological recordings of neurons having Aβ peptide accumulation, demonstrate that the formation of nonspecific ion channels contributes to the neurotoxicity of Aβ peptides and this toxicity may be associated with Alzheimer's disease. Although there is still much debate as to which oligomeric form of Aβ (oligomers or fibrils) is most toxic, increasing evidence indicates that soluble oligomers are more toxic than either monomeric or fibrillar forms of Aβ and that the pore-forming species are Aβ oligomers. In some embodiments of the present teachings, membrane constructs prepared synthetically or derived naturally, for example, planar lipid bilayers, multilamellar liposomes, unilamellar liposomes, unilamellar vesicles, proteoliposomes, micelles, mixed detergent-lipid-micelles, eukaryotic or prokaryotic cell membranes (in fragments or viable whole cells), can be induced to incorporate Aβ peptide ion channels by incubating the membrane construct with Aβ(1-40) or Aβ(1-42) peptides.

### Test Compounds

Test compounds employed in the screening methods of the present teachings include for example, without limitation, synthetic organic compounds, chemical compounds, naturally occurring products, for example, polypeptides, peptides, lipids, polysaccharides, glycolipids, glycoproteins and nucleic acids.

Any chemical compound can be used as a potential inhibitor in the assays of the present teachings. In some embodiments, compounds can be dissolved in an aqueous or an organic (especially dimethyl sulfoxide- or DMSO-based) solvent. In some embodiments, the screening assays are designed to screen large chemical libraries by automating the assay steps. The test compounds can be provided from any convenient source and incubated with one or more membrane constructs per assay well. The assays can be run in parallel (e.g., in microtiter formats on microtiter plates in robotic assays with different test compounds in different wells on the same plate). It will be appreciated that the test compounds which may be suitable candidates or lead compounds capable of inhibiting Aβ peptide ion channel formation and/or activity can be obtained from many suppliers of chemical compounds, including ChemDiv (San Diego, Calif., USA), Sigma-Aldrich (St. Louis, Mo., USA.), Fluka Chemika-Biochemica-Analytika (Buchs Switzerland) and the like.

Inhibiting Aβ peptide ion channel activity as used herein includes any reduction in the functional activity of Aβ peptide ion channels. This includes blocking or inhibiting the activity of the channel or inhibiting the formation of the channel in the presence of, or in response to, an appropriate test compound.

In some embodiments, the high throughput screening methods involve providing a small organic molecule or peptide library containing a large number of potential Aβ peptide ion channel inhibitors. Such “chemical libraries” are then screened in one or more screening assays described herein, to identify those library members (particular chemical species or subclasses) that display a desired characteristic inhibitory activity. The compounds thus identified can serve as conventional “lead compounds” or can themselves be used as potential or actual products.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, organic compounds, including peptides and polypeptides, such as those described in U.S. Pat. Nos. 5,010,175 and 5,641,862 to Rutter et al., and U.S. Pat. No. 5,639,603 to Dower et al; Furka et al., In J. Pept. Prot. Res. 37:487-493 (1991) and Houghton et al., Nature 354:84-88 (1991). In some embodiments, the test compounds can be labeled with a tag, such as a fluorescence, enzyme, protein, or radiolabel. Alternatively, the test compound is not labeled. Other chemistries for generating chemically diverse libraries can also be used. Such chemistries include, but are not limited to: peptoids (e.g., PCT Publication No. WO 91/19735), encoded peptides (e.g., PCT Publication No. WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909 -6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14:309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Kent., USA.; Symphony, Rainin, Woburn, Mass., USA.; 433A Applied Biosystems, Foster City, Calif., USA and 9050 Plus, Millipore, Bedford, Mass., USA.). In addition, numerous combinatorial libraries are themselves commercially available (see, e.g., ComGenex, Princeton, N.J., USA; Asinex, Moscow, Russia; Tripos, Inc., St. Louis, Mo., USA.; ChemStar, Ltd, Moscow, Russia; 3D Pharmaceuticals, Exton, Pa., USA and; Martek Biosciences, Columbia, Md., USA).

The test compounds of the present teachings can encompass numerous chemical classes, though typically they are organic molecules, preferably small organic compounds having a molecular weight of more than 100 and less than about 10,000 daltons, preferably, less than about 2000 to 5000 daltons. Test compounds may comprise functional groups necessary for structural interaction with peptides, particularly hydrogen bonding, and typically include at least an amine, carbonyl, hydroxyl or carboxyl group. The test compounds may comprise cyclical carbon or heterocyclic structures, and/or aromatic or polyaromatic structures substituted with one or more of the above functional groups. Test compounds are also found among biomolecules including peptides, saccharides, fatty acids, steroids, purines, pyrimidines, derivatives, structural analogs or combinations thereof.

In some embodiments, test compounds used in the present teachings can be prescreened to select for test compounds that can bind to Aβ fibrils. The identification and study of test compounds that can bind to Aβ fibrils can be carried out by observing a shift in the inherent UV-Vis or fluorescence spectrum of the test compound upon binding to Aβ fibrils. In some embodiments of the present teachings, the test compound that is capable of binding to Aβ fibrils and methods for determining such binding are described in U.S. Provisional Patent Application Ser. No. 60/940,869 filed May 30, 2007 by the University of California San Diego, entitled “Compounds and Methods For The Diagnosis And Treatment Of Amyloid Associated Diseases” and is referred to and incorporated by reference herein in its entirety.

The determination of Aβ fibril binding by test compounds can be determined, in such cases, on the basis that these molecules may require specific, inherent spectroscopic properties. Radioactivity assays can also be used to study the interaction of the test compounds with Aβ fibrils. In some embodiments, preselection of test compounds that can bind to Aβ fibrils can be identified using binding assays, for example, ELISA-based assays. These ELISA based assays entails the screening of molecules that inhibit the interaction of Aβ fibrils with a monoclonal anti-Aβ IgG raised against residues 3-8 of AD-related Aβ peptide (clone 6E10, Abcam Inc., Cambridge, Mass.). Monoclonal anti-Aβ IgGs can be used in this assay to minimize the possibility for direct binding interaction between the IgGs and the test compounds. Without wishing to be bound to any specific theory, it is believed that the test compounds that can effectively and efficiently coat Aβ fibrils are also able to inhibit the binding of this anti-Aβ IgG to Aβ fibrils. The relative inhibition of IgG-Aβ fibril interactions by test compounds is quantified using a standard ELISA protocol commonly used in the immunological arts.

The experimental details for this assay are similar to those reported for the inhibition of IgG-Aβ fibril interactions with Thioflavin T (ThT) and Diaminoacridine (DAA) is discussed in Inbar, P. et al., Chem Bio Chem, 7:1563-1566, which is hereby incorporated herein in its entirety. Test compounds that efficiently coat the surface of Aβ fibrils and inhibit the IgG-Aβ fibril interactions results in a decrease in the amount of the anti-Aβ IgG bound to the Aβ fibrils in this assay. In some embodiments, this preselection assay can distinguish between molecules that bind and coat Aβ fibrils (e.g., ThT and DAA) and molecules that do not interact with Aβ fibrils (e.g., 1-naphthol-4-sulfonate).

In some embodiments, an illustrative list of test compounds used in the Examples and in Table 1 of the U.S. Provisional Patent Application Ser. No. 60/940,869 filed May 30, 2007 hereby incorporated herein with the present teachings, are commercially available and are believed to interact with Aβ peptides, Aβ oligomers, or amyloid fibrils (Alzheimer's-related, prion-related, or other fibrils). Briefly, these test compounds can be categorized as: a) histological staining agents for amyloid fibrils b) molecules that inhibit fibrilogenesis of Aβ peptides and/or destabilize preformed amyloid fibrils, regardless whether these molecules interact directly with Aβ peptides, Aβ oligomers, or Aβ fibrils, c) a molecule known not to bind to Aβ fibrils (as a negative control) and d) molecules for which their involvement with the treatment or diagnosis of Alzheimer's disease are unknown.

In some embodiments of the present teachings, the test compounds can be any isolated natural or synthetically derived molecule.

### Screening Assays for Inhibitors of Aβ Peptide Ion Channel Activity

The present teachings provide for sensitive, rapid, medium to high-throughput screening assays that permit rapid quantitative and/or qualitative analysis of Aβ peptide ion channel activity using synthetic or natural membrane constructs that have Aβ peptide ion channels embedded in the membrane. Screening assays can comprise different methods for detecting and measuring the pore forming activity of Aβ peptide oligomers when added to a membrane construct. In some embodiments of the present teachings, the ability of a test compound to inhibit either the ability of the Aβ peptide to form pores and ion channels de novo, or inhibit the activity of preformed Aβ peptide ion channels can be determined using direct measurement of ion channel activity using planar lipid bilayers, single wavelength fluorescence correlation spectroscopy and modified whole cell patch-clamp ion channel recording assays.

In some embodiments of the present teachings the screening assays can comprise the steps: providing a membrane construct disposed on a substrate; contacting the membrane construct with Aβ peptide capable of forming an Aβ peptide ion channel in the membrane construct; contacting the membrane construct with a test compound; and determining an Aβ peptide ion channel activity after the construct has incubated with the Aβ peptide in the presence of the test compound; wherein a reduction in the Aβ peptide ion channel activity of the membrane construct contacted with the test compound in comparison to a different membrane construct contacted with the Aβ peptide in the absence of the test compound indicates that the test compound is an inhibitor of amyloidal neurodegenerative disease.

### Direct Measurement of Ion Channel Activity in Planar Lipid Bilayers

In some embodiments of the present teachings, screening assays can employ test chambers or test wells that utilize various multiwell microtiter plate formats, for example, 2 well, 6 well, 8 well, 16 well, 32 well, 64 well, 96 well, 384 well and 1516 well plates for the determination of inhibitory Aβ peptide ion channel compounds also referred to herein as lead compounds. In addition, it is apparent to those of ordinary skill in the art, to adapt these multiwell assays to many different types of high throughput methods, and can include in some embodiments, other solid supports and methods including beads, microarrays and microstamping (Mayer, M. et al., Biophysical Journal, (2003), 85(10):2684-2695; and Mayer et al., Proteomics, (2004), 4:2366-2376).

In some embodiments, the membrane constructs can be placed on substrates that are part of wells or chambers, for example, the bottom of a well of a multiwell microtiter plate that enable direct measurement of ion channel activity of the membrane construct or cells that are disposed on the surface of the substrate. In some embodiments, the substrate can be a solid support. In some embodiments, the solid support can be a well or chamber bottom, as in the well bottom of a microtiter plate. In some embodiments, the solid support can be a solid surface placed on top of a substrate comprising the same or different material that makes up the substrate. In some embodiments, a solid support can be any solid phase material upon which a membrane construct is attached or disposed. A solid support can include natural materials including polysaccharides, complex polysaccharides, proteins, or, they may be composed of synthetic materials including polymeric materials, for example, polypropylene, polyethylene terephthalate (PET), polyester, polystyrene, polycarbonate, polyfluoroethylene, polyethylene, polyacrylamide, silicon wafers, hydrogels, and the like. In some embodiments, the solid support can also include: Teflon, Delrin, glass, polyimide, ceramic, plastic, and the like. In some embodiments, the substrate can be planar, substantially planar, or non-planar, for example, convex, concave and U-shaped.

In some embodiments, the substrate and solid support can be transparent for detection of optical signals used in the screening assays of the present teachings. Alternatively, the substrate and solid supports can be opaque, particularly useful, when the measure of Aβ peptide ion channel activity involved measuring emitted fluorescence.

In some embodiments, the substrate can be a multi-well plate having 2 wells, 6 wells, 8 wells, 16 wells, 32 wells, 64 wells, 96 wells, 384 wells and 1516 wells, each well having a solid support on which to place a membrane construct, including for example, a planar lipid bilayer, a multilamellar liposome, a unilamellar liposome, a unilamellar vesicle, a proteoliposome, a micelle, a mixed detergent-lipid-micelle, a whole cell membrane (as found in viable eukaryotic and prokaryotic cells), a cell membrane fragment and mixtures thereof.

In some embodiments of the present teachings, methods for quantifying Aβ peptide ion channel activity in planar lipid bilayers can be determined by measuring the total transported charge in a given time interval (for example, 1 min.). The quantitative screening assays of the present teachings, can include methods for measuring the Aβ peptide ion channel activity in the presence and absence of a test compound. The activity of the Aβ peptide ion channel can then be averaged over a number of trials at one concentration of Aβ peptide. The Aβ peptide ion channel activity in the planar lipid bilayer can be determined by increasing the concentration of Aβ peptide in the assay and thus, reflect a greater number of Aβ peptide ion channels in the lipid bilayers.

### Fluorescence Correlation Spectroscopy (FCS).

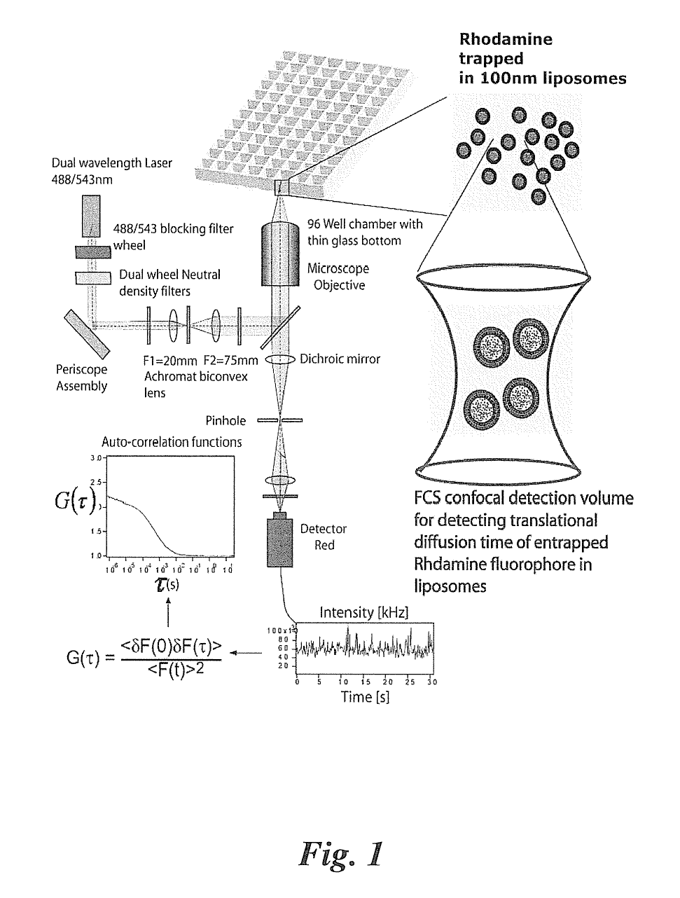

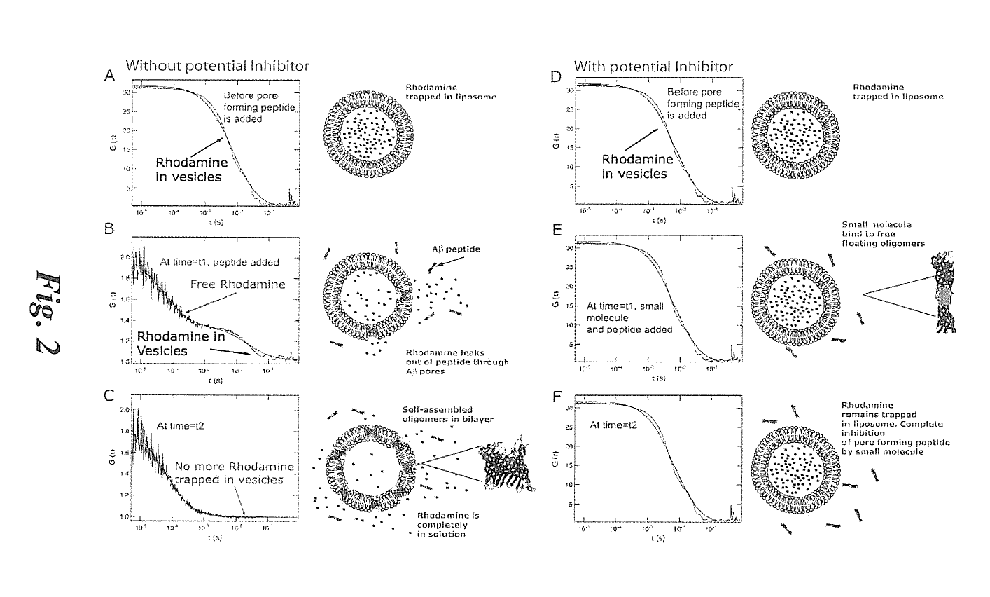

The determination and analysis of fluorophore release or actual ion current measurement across the ion channels provides a basis for identifying test compounds that can disrupt Aβ peptide ion channel activity embedded in the membrane or prevent de novo synthesis of Aβ peptide ion channels from forming. The assay methodology is illustratively shown in FIGS. 1 and 2. Initially, the activity of the Aβ peptide ion channel can be determined by measuring the release of a fluorophore from a fluorophore filled membrane construct, for example, a liposome over a range of time periods, and under varying Aβ peptide concentrations. In some embodiments, a quantitative approach using the screening assays described herein consist of measuring the release of a fluorophore from a membrane construct using the assays of the present teachings as a function of Aβ peptide ion channel activity. When a test compound is found to contribute to a reduction or elimination of fluorophore release from fluorophore filled liposomes having Aβ peptide ion channels, in comparison to the control, having the same type of liposomes similarly filled with the same fluorophore in the absence of the test compound, then the test compound is said to be an inhibitor of Aβ peptide ion channel activity. In some embodiments, screening assays designed to measure the inhibitory activity of a test compound can use highly sensitive medium to high throughput functional screening assays using fluorophore filled liposomes.

In some embodiments, the FCS screening assay can measure the inhibition of diffusion of fluorophore, for example, rhodamine from a liposome, after the liposome has been incubated with Aβ peptide and a test compound either sequentially or simultaneously. Control samples can be prepared where the incubation of rhodamine filled liposomes, with Aβ peptides results in the diffusion of rhodamine from the liposome into the milieu. In some embodiments, the fluorophore, can be any fluorescence or luminescence probe having a molecular size that is small enough to be transported across Aβ peptide ion channels. In some embodiments, the fluorophore can be any suitable fluorescence or luminescence probe including for example: rhodamine, Lucifer Yellow CH Dilitium salt, sodium green, calcein red-orange, Fluo-3, Fluo-4, magnesium orange, magnesium green, indo-1, fura-2 and fura-red. Suitable fluorophores can be found at www.fluorophore.org URL, a site managed by Torsten Mayr, Institute of Analytical Chemistry at the Graz University of Technology, Graz, Austria.

The ratio of mass between free rhodamine and 100 nm diameter liposomes filled with rhodamine is close to 1×10⁶. In some embodiments, the assays employing FCS herein can measure the inhibitory effects of test compounds on pore-forming Aβ peptide activity. In some embodiments, the Aβ peptide ion channel activity can be measured and detected by either slower pore-forming kinetics (partial inhibition) or by lack of release of the fluorophore from the liposomes (complete inhibition).

In some embodiments of the present teachings screening assays to identify lead compounds that are capable of inhibiting Aβ Peptide Ion Channel Activity include the steps: providing a membrane construct filled with a fluorophore disposed on a substrate; contacting the membrane construct with Aβ peptide capable of forming an Aβ peptide ion channel in the construct; contacting the membrane construct with a test compound; and determining an Aβ peptide ion channel activity after the construct has incubated with said Aβ peptide in the presence of the test compound by measuring diffusion of a fluorophore from the membrane construct with a fluorescence detector; wherein a reduction in the Aβ peptide ion channel activity of the membrane construct contacted with the test compound in comparison to a different membrane construct contacted with the Aβ peptide in the absence of the test compound indicates that the test compound is an inhibitor of amyloidal neurodegenerative disease.

### MTT Cell-Toxicity Assays

In some embodiments, the activity of test compounds to inhibit the Aβ peptide ion channel activity or formation of Aβ peptide ion channels in vitro can be determined using a modified MTT colorimetric assay. The MTT assay is an assay that can determine the viability and growth of cultured cells in vitro. In some embodiments, the MTT assay can also be used to determine cytotoxicity of Aβ peptide ion channel activity in cultured neuronal cells. Yellow MTT (3-(4,5-Dimethylthiazol-2-yl)-2,5-diphenyltetrazolium bromide) is reduced to purple formazan in the mitochondria of living cells. A solubilization solution (usually either dimethyl sulfoxide, an acidified ethanol solution, or a solution of the detergent sodium dodecyl sulfate in dilute hydrochloric acid) is added to dissolve the insoluble purple formazan product into a colored solution. The absorbance of this colored solution can be quantified by measuring at a certain wavelength (usually between 500 and 600 nm) by a spectrophotometer.

The chemical reduction takes place only when mitochondrial reductase enzymes are active, and therefore conversion can be directly related to the number of viable (living) cells. When the amount of purple formazan produced by cells treated with an agent is compared with the amount of formazan produced by untreated control cells, the effectiveness of the agent in causing death of cells can be deduced, through the production of a dose-response curve. Methods for performing the MTT assay can be found in Mosmann, T., Rapid Colorimetric Assay for Cellular Growth and Survival: Application to Proliferation and Cytotoxicity Assays. J. Immunol. Meth. (1983), 65, 55-63.

### Patch-Clamp Assays for Identifying Compounds Capable of Inhibiting Aβ Peptide Ion Channel Activity in Living Cells

In some embodiments, the inhibitory effect of the test compound against Aβ peptide ion channel activity can be measured using patch-clamp electrophysiological measurement using high-throughput whole neuronal cell recordings. In some embodiments, a microtiter plate having at least 96 wells, for example, a 384 well or 1536 well microtiter plate can be used to prepare screening assay which can be used to determine whether a test compound (for example, in a sample of one, to tens, to hundreds or a library of test compounds) can inhibit neurotoxic Aβ peptide ion channel activity can be designed to include an array which comprises a plurality of samples containing neuronal cells and Aβ peptides in the presence or absence of one or more test compounds. The cells can be immobilized on a multi-well plate, well or chamber, or solid support and be in contact with an electrode reading device capable of measuring Aβ peptide ion channel activity in each cell immobilized on different extracellular potential-sensitive electrodes.

In some embodiments, once electrical access to the cell interior is achieved, the whole cell patch-clamp device, for example an “IONWORKS”® originally developed by Essen Technologies, Ann Arbor, Mich.; USA is able to voltage clamp the membrane construct in each respective well, thereby enabling electrophysiological recordings. In some embodiments, the applied voltage waveform to be applied across the membrane construct to determine the Aβ peptide ion channel activity can depolarize the cells from a resting potential of −100 mV to 0 mV to 100 mV for approximately 100 msec. Upon depolarization, a small inward current measuring on the order of a few nA (10⁻⁹ Amp) is present with the characteristic time signature of Aβ peptide ion channel recordings.

In some embodiments, the screening assay using a whole cell patch-clamp assay of the present teachings can involve a number of sequences to determine the effect of a test compound on the Aβ peptide ion channel activity. In one embodiment, a pre-test compound sequence involves a measurement sequence comprising the taking of a pre-test compound recording from each well, followed by the addition of a one or more test compounds by the device's fluidics system or manual application. In some embodiments, to ensure a valid measurement, a particular test compound is added to multiple wells of the multi-well substrate and can be added at varying concentrations to obtain a dose response curve for the particular test compound being screened. This provides some system redundancy at the expense of compound throughput, i.e., the number of different test compounds that can be analyzed per day.

After a short incubation time (during which the test compound is in the presence of the membrane construct, typically 3-5 minutes), the device electronics system can revisit the substrate, initiating the same recording sequence to measure the effect of each test compound on a post-compound recording. Measurements, in this manner allow the direct comparison of the same membrane construct, preferably a viable cell, before and after the addition of the test compound. This makes the measurement “differential” in nature, allowing for good assay performance even in the presence of widely varying individual ion channel current levels, as each well can serve as its own control. In addition, differential measurements offer the advantage, in the case of the present Aβ peptide ion channel activity inhibition-type assays, to discriminate between cells that have particular ion channel current expression and those that do not. Cells without expression on the pre-test compound recording thus can be excluded from post-analysis. In some cases, the test compound or test compounds may be added first, followed by a single electrical measurement read.

In some embodiments of the present teachings, screening assays employing whole cell patch-clamp ion activity recording in the presence and absence of test compounds can comprise the steps: providing a membrane construct disposed on a substrate; contacting the membrane construct with Aβ peptide capable of forming an Aβ peptide ion channel in the construct; contacting the membrane construct with a test compound; and determining an Aβ peptide ion channel activity after the construct has incubated with said Aβ peptide in the presence of the test compound by measuring Aβ peptide ion channel activity of the membrane construct with a high-throughput whole cell patch-clamp recording device; wherein a reduction in the Aβ peptide ion channel activity of the membrane construct contacted with the test compound in comparison to a different membrane construct contacted with the Aβ peptide in the absence of the test compound indicates that the test compound is an inhibitor of amyloidal neurodegenerative disease.

Such high-throughput, multiplexed patch-clamp whole cell recoding devices are commercially available. Suitable examples of such devices can include: “IonWorks” device manufactured by Essen Technologies, Ann Arbor, Mich.; USA; and PatchXpress® 7000A Automated Parallel Patch-Clamp System available from Molecular Devices Corp., Sunnyvale, Calif., USA.

Specific Examples are provided for illustrative purposes of how to make, use and practice the compositions and methods of this technology and, unless explicitly stated otherwise, are not intended to be a representation that given embodiments of this technology have, or have not, been made or tested.

### Examples

### Example 1

### Fluorescence Correlation Spectroscopy (FCS) Assay

In the various assays, Aβ peptide in the form of Aβ(1-42) peptide or Aβ(1-40) peptide can be used to form self-aggregated fibrils capable of forming ion channels in membrane constructs. In some embodiments, the Aβ fibrils are grown from synthetic Aβ (1-42) peptides by incubating the peptides (74 μM) in ultrapure water at 37° C. for 72 hours. Fibrils are characterized by electron and scanning probe microscopy. (P. Inbar, J. Yang, Bioorg. Med. Chem. Lett. 2006, 16(4), 1076-1079).

Liposomes filled with rhodamine can be prepared by forming small liposomes using an extruder for obtaining a narrow size distribution of liposomes). Free (non-entrapped) rhodamine can be eliminated by buffer exchange using Microspin S-200 HR columns. Rhodamine-filled liposomes can then be added to a 96, 384 or a 1536 multi-well plate with a glass cover slip base (Whatman® cat#: 7706-1365). The apparatus and laser selection for emission and fluorescence detector for recording observed liposome fluorescence are illustrated in FIG. 1. To switch rapidly between all wells, Aβ peptides at concentrations ranging from 10 nM to 40 μM can be added to each well and can be read for ≦1 second to quantify the leakage of rhodamine out of liposomes. A computer-controlled X,Y,Z-microscope stage is installed to switch rapidly between wells in a programmable or parallel fashion.

Test compounds that are capable of inhibiting or reducing the kinetics of fluorophore release (partial inhibition) or complete inhibition of fluorophore release (complete inhibition) from the fluorophore filled liposomes incubated previously with or concurrently with Aβ peptides as compared to a control well comprising the same assay conditions minus the test compound, the test compound will be identified as a lead compound and an inhibitor of amyloidal neurodegenerative disease. FIG. 2. illustrates the release of rhodamine from 100 nm diameter liposomes incubated with Aβ peptide oligomers, and inhibition of rhodamine release by the presence of a test compound having an inhibitory effect against the same Aβ peptide oligomers. As can be seen when comparing panels C (no test compound, referred to as small molecule) and F (complete inhibition of rhodamine release due to the incubation of the test compound with the Aβ peptide oligomers.

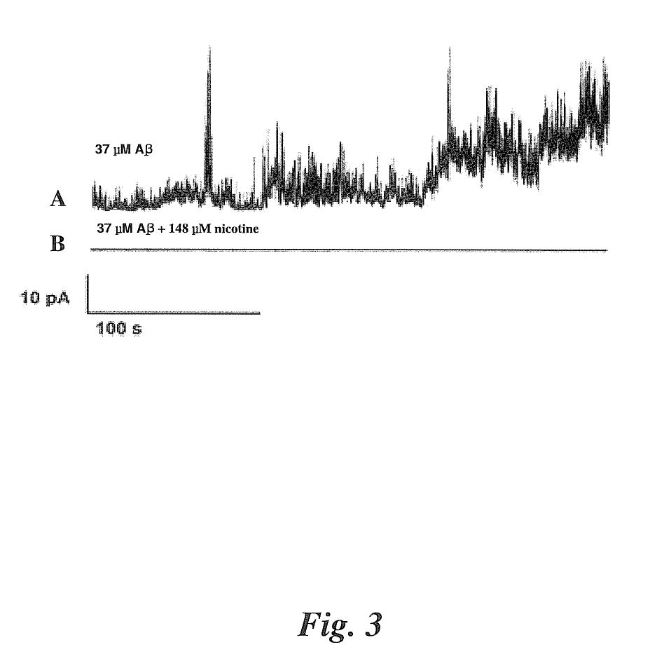

For screening large numbers of test samples, the first analyses can be performed using previously identified test compounds that were found to inhibit Aβ pores using electrophysiological screening assay presented in FIGS. 3-5. These molecules include: nicotine, Congo Red, tannic acid, dopamine, as well as derivatives of these molecules synthesized by Prof J. Yang at the University of California San Diego (UCSD) and disclosed in U.S. Provisional Patent Application Ser. No. 60/940,869, filed May 30, 2007 entitled “Compounds and Methods For The Diagnosis And Treatment Of Amyloid Associated Diseases” and is incorporated herein in its entirety. These test compounds can be tested at molar ratios of Aβ peptide to test compound of 1:0.1, 1:1, 1:10, and 1:100. Incubation of the test compound can be performed in at least the following ways: i) Aβ and candidate inhibitor can be pre-incubated and then added to the liposome solution; ii) test compound is added first to the liposomes in the 96-well plates followed by addition of Aβ oligomers. In general, test compounds that inhibit ion channel formation by pore-forming Aβ peptides can act in various ways, without wishing to be bound by theory, these examples include test compounds that bind to membranes and compete with Aβ for “binding sites”, test compounds that disrupt pore-forming oligomers, test compounds that block pores, and combinations of these mechanisms. For each test compound or panel of test compounds performed in a single assay, a negative control without Aβ will be performed to detect those molecules that may have fluorescent properties or that may disrupt liposome membranes.

### Example 2

### Electrophysiological Lipid Bilayer Assay for Aβ Peptide Ion Channel Activity Using Bilayer Chambers

Ion channel measurements. Screening assays can be performed using single channel recordings in “voltage clamp mode” using Ag/AgCl electrodes (Warner Instruments) in each compartment of the bilayer chambers. Data acquisition and storage can be carried out using custom software in combination with either an EPC-7 patch clamp amplifier from List Medical Electronic (set at a gain of 10 mV pA-1 and a filter cutoff frequency of 3 kHz) or a Geneclamp 500 amplifier from Axon Instruments (with a CV-5B 100GU headstage, set at a gain of 100 mV pA-1 and filter cutoff frequency of 1 kHz). In some embodiments, patch clamp amplifiers, for example, the EPC-7 amplifier can be used for most folded bilayers and the Geneclamp 500 amplifier for most painted bilayers. The data acquisition boards for both amplifiers were set to a sampling frequency of 15 kHz. Current traces can be further filtered using a digital Gaussian low-pass filter with a cutoff frequency of 30 Hz. In some embodiments, the current traces used to generate data that can be recorded at applied potentials 50 mV and filtered at 10 Hz.

Analysis of the single channel current traces can be made by computing histograms of the currents from the original current-time traces with ClampFit 9.2 software from Axon Instruments. From these histograms, the main current values can be extracted by fitting a Gaussian function to the peaks in the histograms. Single channel conductances can refer to the main conductance state (i.e. to the dominant peaks in the current histograms).

The lipid mixture can be made from POPE:POPG (Avanti Polar Lipids) at 25 mg/ml (1:1) in Heptane. The pretreatment lipid solution can be POPE:POPG 20 mg/mL in Hexane. 2. The bilayer is formed in classic bilayer cups and chamber (Warner Instruments). This 2-part system consists of a black Delrin chamber and a cup of Delrin. Cups and chambers are designed such that addition of equal volumes to the cup and chamber (cis and trans sides) results in a balanced solution height, minimizing any pressure gradients across the bilayer membrane.

The bilayer was formed over a 250-μm hole in a partition separating two Delrin compartments, the so called “painting technique.” First, droplets of pretreated lipid solution were placed on both sides of the hole, using 100 μL-Hamilton syringe. Once the droplet of hexane evaporates, both chambers (cis and trans) were symmetrically filled with 900 μL of buffer solution, 100 mM KH₂PO₄/K₂HPO₄ pH 7.4, at room temperature.

When the bilayer set-up was already in place with Ag/AgCl electrodes in Faraday cage (a bilayer workstation which provides critical shielding of electromagnetic interference from outside sources and isolate vibration) the lipid solution (POPE:POPG in Heptane) can be painted directly over the hole by using a thin paintbrush. Blowing air underneath the hole using a Pasteur pipette to thin out the bilayer can be used until the appropriate capacitance (80-120 pF) is achieved. A voltage of ±100 mV was applied for at least 10 minutes to test stability of lipid bilayer.

A time-averaging method is used to measure and quantify the ion channel current from ion channel forming as shown previously for antibiotic peptides in planar lipid bilayers (Blake, S., et al., Chem Bio Chem 7, 433-435 (2006); Mayer, M., et al., in preparation (advanced draft). This approach is adapted to the analysis of Aβ peptide ion channel activity. Aβ peptides (1-42 and 1-40) are commercially available from Bachem Bioscience Inc., (King of Prussia, Pa., USA). Solubilizing agents (e.g. DMSO, TFA, or TFE) (Walsh, D. M., et al., Biochem. J. (2001), 355:869-877) are then used to ensure all Aβ peptides are present as monomers and not in pre-aggregated state of oligomers and fibrils.

Aβ(1-42) (commercially available from Innovagen, Lund, SE or Bachem Bioscience Inc., King of Prussia, Pa., USA) is initially dissolved in deionized water at 1 mg/ml (221.5 μM) and stored at −20° C. The stock solution can be aliquotted to sufficient amount for each time use (90 μL). After the stable bilayer is constituted, the Aβ(1-42) solution can be added to the trans side of the chamber to obtain a final concentration of 37 μM. The solution is mixed well in the chamber under stirring for 5 minutes.

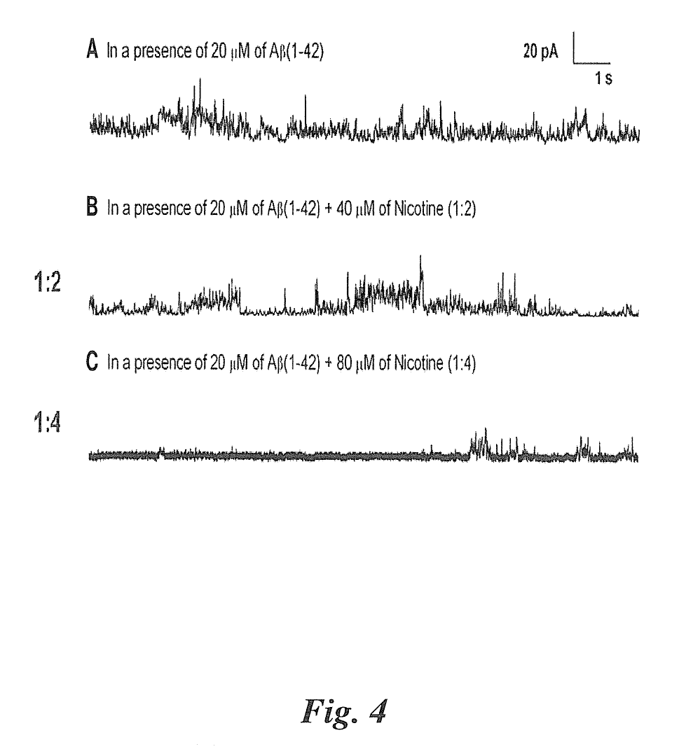

Congo Red (Sigma) is dissolved in de-ionized (DI) water to achieve a concentration of 2.5 mg/ml (3.58 mM as a stock solution). (−)Nicotine hemisulfate (Sigma, St. Louis, Mo., USA) was diluted to 1.89 mM in DI water. Tannic acid (Riedel-de Haën, Seelze, Germany) is diluted to 20 mM in DI water. The solution of inhibitory molecule/test compound is added to cis and trans sides to make a desired final concentration (1:1 molar ratio) at the same time as the addition of the Aβ peptide. As shown in FIGS. 3 and 4, using nicotine, the solution was added after observing current activities of Aβ peptide to obtain 1:1, 1:2, 1:3, and 1:4 molar ratio. FIG. 3 shows that the addition of nicotine resulted in concentration-dependent disruption of Aβ peptide ion channel activity. A molar ratio of nicotine to Aβ peptides of 4:1 disrupted preformed Aβ peptide ion channel activity almost completely. Control experiments with molecules known not to bind to Aβ did not result in inhibition of Aβ ion channel activity. It was observed that the disruption of preformed Aβ ion channels required more nicotine than the inhibition de novo formed ion channels.

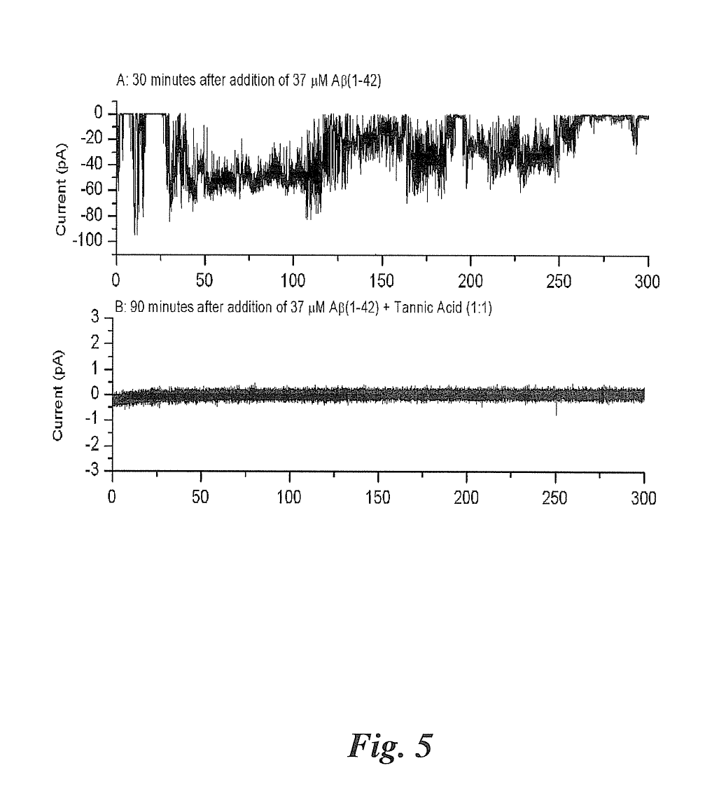

FIG. 5 was repeated with Tannic acid. Tannic acid and Congo Red (not shown) had been found to bind strongly to aggregated Aβ fibrils. Tannic acid was shown to inhibit ion channel activity of Aβ.

To quantify the amount of inhibition, the screening assays are designed to measure the amount of current traversing through the Aβ ion channels. A first set of experiments may be carried out with Aβ(1-42) peptide as this peptide is important for the neurotoxic mechanism of the disease and is known to form significant ion channel activity in planar lipid bilayers as well as in the membrane of living cells. Aβ(1-40) peptide shares these characteristics but it aggregates more slowly into fibrils and it takes longer before ion channel activity is observed.

The ion channel activity of Aβ peptide is quantified by measuring the total transported charge in a given time interval (e.g. 1 min). This experiment is performed multiple times, for example, at least four times, to obtain a reliable average and then repeated at increasing concentrations of the Aβ(1-42) peptide, while keeping all other parameters constant. The present screening assay will yield reliable statistics to establish the total transported charge through Aβ ion channels as a function of the concentration of Aβ peptide. (Blake, S., et al., Chem Bio Chem 7, 433-435 (2006); Mayer, M., et al., in preparation (advanced draft) (2006); Mayer, M., et al., Proteomics 4, 2366-2376 (2004).) The possible dependence of the ion channel activity on the time-lapse after the addition of the Aβ peptides is analyzed. A well-established time-dependence will make it possible to compare the ion channel activity before and after addition of a test compound (small molecules) with binding affinity for Aβ fibrils. The results obtained with Aβ(1-42) peptide may be compared with the results obtained with Aβ(1-40) peptide. All recordings are carried out at 37° C. using a microfabricated planar lipid bilayer setups, which afford reliable and stable low-noise recording conditions. These setups have been developed and optimized. (Blake, S., et al., Chem Bio Chem 7, 433-435 (2006); Mayer, M., et al., in preparation (advanced draft) 2006); Mayer, M., et al., Eur. Biophys. J. Biophys. Lett. 29, 378 (2000); Terrettaz, S., et al., Langmuir 19, 5567-5569 (2003); Mayer, M. in Physical Chemistry 25-61 (Swiss Federal Institute of Technology, Lausanne, Switzerland, 2000); Schmidt, C. et al., Chemie Int. Ed. 39, 3137-3140 (2000); Mayer, M., et al., in Biosensors: A practical approach (eds. Cooper, J. M. & Cass, A. E. G.) 153-184 (Oxford University Press, Oxford, 2003) and Mayer, M., et al., Biophys. J. 85, 2684-95 (2003)).

Using the present quantitative ion channel assay, the ion channel activity of Aβ peptides before and after addition of the test compound is compared. Dose-response curves of inhibition of ion channel activity as a function of increasing concentrations of test compound are constructed. The inhibitory effect of the test compounds that are capable of interfering with the assembly process of Aβ peptides to ion channels have been found to typically follow a power law with respect to the concentration of the inhibitory test compound. (Mayer, M., et al., in preparation) The inhibitory effect thus is expected to increase strongly (non-linearly) with concentration.

### Example 3

### High-Throughput Patch Clamp Neuronal Cell Based Screening Assay

Test compounds can be screened to identify lead compounds that are capable of inhibiting Aβ peptide ion channel activity. Test compounds can be assayed by adding different concentrations of test compound to different substrates, each having a substantially similar population of human neuronal SH-SY5Y neuronal cells (human neuroblastoma cells). The test compound can be selected from known compounds that have been shown to bind to Aβ peptides, oligomers or Aβ fibrils, or unknown test compounds not having been previously characterized.

The screening assays are prepared by first growing SH-SY5Y neuronal cells that have been incubated and grown in cell culture medium containing Aβ peptides for several days. The toxicity of the Aβ peptides on the neuronal cell line can be determined using an MTT assay as developed by Bollimuntha et al., Brain Res. (2006) 1099:141-149. At least one test well and several controls can be arranged in an array format. In parallel, wells are prepared containing the SH-SY5Y cells, Aβ peptides, and a test compound to determine whether the test compound has an effect on Aβ peptide ion channel activity. In addition to the two wells, one with test compound, and one without, two added controls can be prepared. The first control includes the neuronal cells mixed with growth medium only, and the second control includes cells, growth medium, and the test compound, but no Aβ peptide added.

The inhibitory effect of the test compound against Aβ peptide ion channel activity can be measured using patch-clamp electrophysiological measurement using high-throughput whole cell recordings. An array can be prepared which comprises a multiplicity of neuronal cells immobilized on a substrate or solid support or electrode in contact with an electrode reading device capable of measuring ion channel activity in each cell immobilized on different extracellular potential-sensitive electrodes. In certain embodiments, the array can accommodate up to 384 samples including a plurality of test compounds and controls (i.e. one such control, samples of neuronal cells having Aβ peptides added but not test compound) to be used for high throughput screening of ion channel activity in parallel. Useful high-throughput devices for measuring the ion channel activity of the present cells can include the electrophysiological recording device known as the “IonWorks®” device manufactured by Essen Technologies, Ann Arbor, Mich., USA. The IonWorks® device and methods of using the device is described in U.S. Pat. No. 7,270,730, Ser. No. 10/236,684 to Schroeder et al., and is incorporated by reference herein in its entirety.

The IonWorks® instrument can be an integrated platform that consists of computer-controlled fluid handling, recording electronics, and processing tools capable of voltage clamp whole-cell recordings from hundreds to thousands of individual cells. To establish a recording, the system can be a planar, multiwell substrate including, but not limited to, a PatchPlate, including high throughput 384 well plates, each well with at least one aperture. The system can effectively position 1 cell into a perforation separating 2 fluid compartments in each well of the substrate. Voltage control and current recordings from the cell membrane are made subsequent to gaining access to the cell interior by applying a permeabilizing agent to the intracellular side. Based on the multiwell design of the PatchPlate, voltage clamp recordings of up to 384 individual cells can be made in minutes and are comparable to measurements made using traditional electrophysiology techniques.

The comparison of cellular Aβ peptide ion channel activity between the samples of neuronal cells having Aβ peptide ion channels in the presence of a test compound to samples containing neuronal cells with Aβ peptide ion channels but no test compound can establish whether the test compound can inhibit Aβ peptide ion channel activity and if so, the test compound is therefore a lead compound and an inhibitor of amyloidal neurodegenerative disease.

What is claimed is:

1. A screening assay for screening potential inhibitors of amyloidal neurodegenerative disease comprising:
providing a membrane construct disposed on a substrate;
contacting said membrane construct with Aβ peptide capable of forming an Aβ peptide ion channel in said construct;
contacting said membrane construct with a test compound; and
determining an Aβ peptide ion channel activity after said construct has incubated with said Aβ peptide in the presence of said test compound;
wherein a reduction in said Aβ peptide ion channel activity of said membrane construct contacted with said test compound in comparison to a different membrane construct contacted with said Aβ peptide in the absence of said test compound indicates that the test compound is an inhibitor of amyloidal neurodegenerative disease.

2. The assay according to claim 1, wherein said determining an Aβ peptide ion channel activity comprises measuring an ion current across an ion channel comprising Aβ peptide oligomers in said membrane construct.

3. The assay according to claim 2, wherein measuring an ion current across an ion channel comprises measuring the total transported charge through said Aβ peptide ion channel over a given time period.

4. The assay according to claim 2, wherein said membrane construct comprises a planar lipid bilayer, a multilamellar liposome, a unilamellar liposome, a unilamellar vesicle, a proteoliposome, a micelle, a mixed detergent-lipid-micelle, a whole cell membrane, a eukaryotic cell, a prokaryotic cell, a cell membrane fragment and mixtures thereof.

5. The assay according to claim 4, wherein said membrane construct is a planar lipid bilayer.

6. The assay according to claim 1, wherein said determining an Aβ peptide ion channel activity comprises determining an Aβ peptide ion channel activity after said construct has incubated with a test compound prior to adding said Aβ peptide to said membrane construct

7. The assay according to claim 1, wherein the Aβ peptide comprises Aβ peptide (1-40) or Aβ peptide (1-42).

8. A screening assay for screening potential inhibitors of amyloidal neurodegenerative disease comprising:
providing a membrane construct filled with a fluorophore disposed on a substrate;
contacting said membrane construct with Aβ peptide capable of forming an Aβ peptide ion channel in said construct;
contacting said membrane construct with a test compound; and
determining an Aβ peptide ion channel activity after said construct has incubated with said Aβ peptide in the presence of said test compound by measuring diffusion of a fluorophore from said membrane construct with a fluorescence detector;
wherein a reduction in said Aβ peptide ion channel activity of said membrane construct contacted with said test compound in comparison to a different membrane construct contacted with said Aβ peptide in the absence of said test compound indicates that the test compound is an inhibitor of amyloidal neurodegenerative disease.

9. The assay according to claim 8, wherein the membrane construct is a liposome.

10. The assay according to claim 8, wherein measuring said diffusion of a fluorophore within said membrane construct comprises measuring the fluorescence of said membrane construct in the presence and absence of a test compound using an excitation wavelength specific for said fluorophore.

11. The assay according to claim 8, wherein said fluorophore comprises rhodamine, Lucifer Yellow CH Dilitium salt, sodium green, calcein red-orange, Fluo-3, Fluo-4, magnesium orange, magnesium green, indo-1, fura-2 and fura-red.

12. The assay according to claim 11, wherein said fluorophore is rhodamine.

13. A screening assay for screening potential inhibitors of amyloidal neurodegenerative disease comprising:
providing a membrane construct disposed on a substrate;
contacting said membrane construct with Aβ peptide capable of forming an Aβ peptide ion channel in said construct;
contacting said membrane construct with a test compound; and
determining an Aβ peptide ion channel activity after said construct has incubated with said Aβ peptide in the presence of said test compound by measuring Aβ peptide ion channel activity of said membrane construct with a high-throughput whole cell patch-clamp recording device;
wherein a reduction in said Aβ peptide ion channel activity of said membrane construct contacted with said test compound in comparison to a different membrane construct contacted with said Aβ peptide in the absence of said test compound indicates that the test compound is an inhibitor of amyloidal neurodegenerative disease.

14. The assay according to claim 13, wherein said high-throughput whole cell patch-clamp recording device includes a multi-well plate having up to 384 substrates for recording said Aβ peptide ion channel activity in up to 384 cells in parallel.

15. The assay according to claim 13, wherein said membrane construct is a cell cultured brain cell, a transgenic primary neuron or a primary wild-type neuronal cell.

16. The assay according to claim 15, wherein said transgenic primary neuron comprises isolated primary neurons from embryonic hAPP/hPS-1 transgenic mice.

17. The assay according to claim 15, wherein said human neuronal cell line is a human SH-SY5Y neuronal cell line.

18. The assay according to claim 1, wherein contacting said membrane construct with a test compound comprises contacting said membrane construct with a test compound capable of binding to Aβ peptide.

19. The assay according to claim 1, wherein said substrate is a multiwell plate comprising 2 wells, 6 wells, 8 wells, 16 wells, 32 wells, 64 wells, 96 wells, 384 wells or 1536 wells.

20. The assay according to claim 1, wherein the amyloidal neurodegenerative disease comprises Alzheimer's Disease, cerebrovascular amyloidosis and Lewy body dementia.
